(19) United States
(12) Patent Application Publication
(10) Pub. No.: US 2025/0381366 A1
(43) Pub. Date: Dec. 18, 2025

(54) Customizable Sensory Device for Managing Sensory Processing Disorders

(71) Applicant: Monica De Leon, Tucson, AZ (US)

(72) Inventor: Monica De Leon, Tucson, AZ (US)

(21) Appl. No.: 19/235,955

(22) Filed: Jun. 12, 2025

Related U.S. Application Data
(60) Provisional application No. 63/659,375, filed on Jun. 13, 2024.

(51) Int. Cl. A61M 21/02; A61M 21/00; A63H 3/00; A63H 3/02

(52) U.S. Cl. CPC A61M 21/02; A63H 3/003; A63H 3/02; A61M 2021/0016; A61M 2021/0022; A61M 2021/0027; A61M 2021/0066; A61M 2205/3303; A61M 2205/8206

(57) ABSTRACT

A multifunctional sensory device designed to aid individuals with sensory processing disorders (SPD) in achieving self-regulation is disclosed. The device can be in the form of a plush animal or a backpack. The device includes a plurality of removable weights to provide deep pressure, a scent emitter for calming or alerting smells, a Bluetooth speaker for producing calming sounds, and an embedded vibration coil for adjustable tactile and heating stimulation. The device also features various textured surfaces and modular components for customizable sensory input. Additionally, a connected system monitors and adjusts sensory inputs based on user feedback.

### CROSS-REFERENCE TO RELATED APPLICATION

The present application claims priority to, and the benefit of, U.S. Provisional Application No. 63/659,375, which was filed on Jun. 13, 2024, and is incorporated herein by reference in its entirety.

### FIELD OF THE INVENTION

The present invention generally relates to the field of sensory integration aids. More specifically, the present invention pertains to a multifunctional sensory device designed to assist individuals with Sensory Processing Disorder (SPD) in achieving self-regulation. The device includes adjustable shoulder straps, removable weights, and various textured surfaces. The device features a scent emitter, a Bluetooth speaker for calming sounds, and an embedded vibration coil for tactile stimulation. The device offers customizable sensory inputs and practical storage, providing reliable support and enhanced well-being for individuals with SPD. Accordingly, this disclosure makes specific reference thereto the present invention. Nonetheless, it is to be appreciated that aspects of the present invention are also equally applicable to other like applications, devices, and methods of manufacture.

### BACKGROUND

By way of background, Sensory Processing Disorder (SPD) is a condition characterized by the brain's difficulty in receiving and responding to information that comes through the senses. SPD most commonly impacts touch, hearing, and sight. Individuals with SPD may exhibit a wide range of symptoms, which can manifest as hypersensitivity (over-responsiveness) or hyposensitivity (under-responsiveness) to sensory stimuli such as touch, sound, sight, smell, taste, and movement.

Common behavioral symptoms of SPD include heightened anxiety and stress, frequent meltdowns or tantrums, challenges with attention and focus, reduced social interaction, and avoidance of sensory-rich environments. These behavioral issues can significantly impact daily functioning and quality of life. Physical symptoms may include coordination problems, insomnia, and feeding issues, further complicating the management of the disorder.

Parents and caregivers often face substantial challenges in managing SPD symptoms, which can be both difficult and stressful. Effective management of SPD typically requires a combination of sensory integration strategies and therapeutic interventions aimed at helping individuals achieve better sensory regulation. Current solutions do not provide an easy and cost-effective management for SPD for people of all ages. People desire a device to aid individuals with SPD in achieving self-regulation through various customizable sensory inputs.

Therefore, there exists a long-felt need in the art for an effective solution to help individuals with Sensory Processing Disorder (SPD). There is also a long-felt need in the art for a multifunctional sensory device that is versatile enough to cater to both children and adults with SPD. Additionally, there is a long-felt need in the art for a sensory device that is both engaging and non-threatening for users, offering a comforting and practical tool for managing sensory challenges. Moreover, there is a long-felt need in the art for a device that can be easily customized with interchangeable features such as weights, textures, scent emitters, and auditory stimuli to meet individual sensory needs. Further, there is a long-felt need in the art for a durable and portable sensory device that can be used in various settings, including home, school, and travel. Furthermore, there is a long-felt need in the art for a sensory device that provides a range of sensory inputs to help users with SPD regulate their sensory processing effectively. Finally, there is a long-felt need in the art for a sensory device that not only aids in self-regulation but also offers practical features to enhance its usability and convenience.

The subject matter disclosed and claimed herein, in one embodiment thereof, comprises a multifunctional sensory device designed to assist individuals with Sensory Processing Disorder (SPD) in achieving self-regulation through various sensory strategies. The device can take the form of a plush animal or a backpack and includes a pair of arms and legs equipped with fasteners for secure attachment, allowing the device to be carried comfortably. The device features adjustable shoulder straps with buckles for a customizable fit and a zippered pocket for storing personal items and sensory tools. The device incorporates a plurality of removable weights to provide deep pressure, which is calming for individuals with SPD. Additionally, the device includes at least one scent emitter that can be customized to provide calming or alerting smells, enhancing the sensory experience. The device is equipped with a Bluetooth speaker for producing soothing sounds and a vibration coil embedded within the body for generating adjustable gentle vibrations and heating effects.

In this manner, the multifunctional sensory device of the present invention accomplishes all of the foregoing objectives and provides users with a novel solution for managing Sensory Processing Disorder. The device offers customizable sensory inputs, including tactile, olfactory, auditory, and proprioceptive stimuli, to help users achieve self-regulation and improve their overall well-being. The inclusion of removable weights, various textures, and scent emitters allows for a tailored sensory experience, while the Bluetooth speaker and vibration coil provide additional calming effects. The adjustable shoulder straps and secure fasteners provide comfort, making the device suitable for both children and adults.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a multifunctional sensory device for assisting individuals with sensory processing disorders in achieving self-regulation. The device comprises a body in the form of a plush animal, a pair of arms are attached to the body, each arm has a distal end with hook and loop fasteners for removably attaching the arms to each other, a pair of legs are attached to the body, each leg has a distal end with hook and loop fasteners for removably attaching the legs to each other, a pair of shoulder straps extend from shoulders of the body to diagonally opposite hips, each shoulder strap has a buckle for fastening and adjusting the strap, a plurality of removable weights are configured to be added to the body to provide deep pressure, at least one scent emitter is configured to provide calming or alerting smells, a zippered pocket on the rear surface of the body stores personal items and sensory items, a Bluetooth speaker is configured to produce calming sounds, a vibration coil is embedded within the body for producing adjustable gentle vibrations, and a plurality of pockets are disposed in the body for placing various calming items.

In yet another embodiment, a multifunctional sensory device is disclosed. The device comprises a body in the form of a backpack, a pair of shoulder straps extending from the rear surface of the body, each strap has a buckle for fastening and adjusting the strap, and a plurality of removable weights are configured to be added to the body to provide deep pressure, a plurality of textured surfaces on the body, including rough, soft, and stringy textures, at least one scent emitter is configured to provide calming or alerting smells, a zippered pocket on the rear surface of the body stores personal items and sensory items, a Bluetooth speaker is configured to produce calming sounds, and a vibration coil is embedded within the body for producing adjustable gentle vibrations.

In another embodiment, the removable weights include sand-filled pouches, bead-filled pouches, and weighted discs. In still another embodiment, the scent emitter is selected from the group consisting of a pouch, a clip-on device, and an infused fabric insert. In another aspect, the vibrations are automatically adjusted based on feedback from the user's body. In yet another aspect, a multifunctional sensory toy is disclosed. The toy comprises a pair of arms and a pair of legs, a pair of shoulder straps, a plurality of removable weights configured to provide deep pressure, at least one scent emitter configured to provide calming or alerting smells, a zippered pocket for storing personal items and sensory items, a speaker configured to produce calming sounds and a vibration coil embedded within the body for producing adjustable gentle vibrations. The vibration coil is also adapted to provide heat to the user to provide a soothing effect.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front-perspective view of the multifunctional sensory toy of the present invention in accordance with the disclosed structure;

FIG. 2 illustrates a rear perspective view showing the back side of the multifunctional sensory toy of the present invention in accordance with the disclosed structure;

FIG. 3 illustrates another perspective view of the multifunctional sensory toy of the present invention showing internal components in accordance with the disclosed structure;

FIG. 4 illustrates a perspective view of a user carrying the sensory toy of FIG. 1 as a backpack in accordance with the one embodiment of the present invention;

FIG. 5 illustrates a rear perspective view showing the user carrying the sensory toy as a backpack in accordance with the disclosed structure; and

FIG. 6 illustrates a perspective view of another embodiment of the multifunctional sensory device of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an effective solution to help individuals with Sensory Processing Disorder (SPD). There is also a long-felt need in the art for a multifunctional sensory device that is versatile enough to cater to both children and adults with SPD. Additionally, there is a long-felt need in the art for a sensory device that is both engaging and non-threatening for users, offering a comforting and practical tool for managing sensory challenges. Moreover, there is a long-felt need in the art for a device that can be easily customized with interchangeable features such as weights, textures, scent emitters, and auditory stimuli to meet individual sensory needs. Further, there is a long-felt need in the art for a durable and portable sensory device that can be used in various settings, including home, school, and travel. Furthermore, there is a long-felt need in the art for a sensory device that provides a range of sensory inputs to help users with SPD regulate their sensory processing effectively. Finally, there is a long-felt need in the art for a sensory device that not only aids in self-regulation but also offers practical features to enhance its usability and convenience.

The present invention, in one exemplary embodiment, is a multifunctional sensory device. The device comprises a body in the form of a backpack, a pair of shoulder straps extending from the rear surface of the body, each strap has a buckle for fastening and adjusting the strap, and a plurality of removable weights are configured to be added to the body to provide deep pressure, a plurality of textured surfaces on the body, including rough, soft, and stringy textures, at least one scent emitter is configured to provide calming or alerting smells, a zippered pocket on the rear surface of the body stores personal items and sensory items, a Bluetooth speaker is configured to produce calming sounds, and a vibration coil is embedded within the body for producing adjustable gentle vibrations.

Reference will now be made in detail to the present preferred embodiments of the invention, examples of which are illustrated in the accompanying drawings. Wherever possible, the same reference numerals are used in the drawings and the description to refer to the same or like parts.

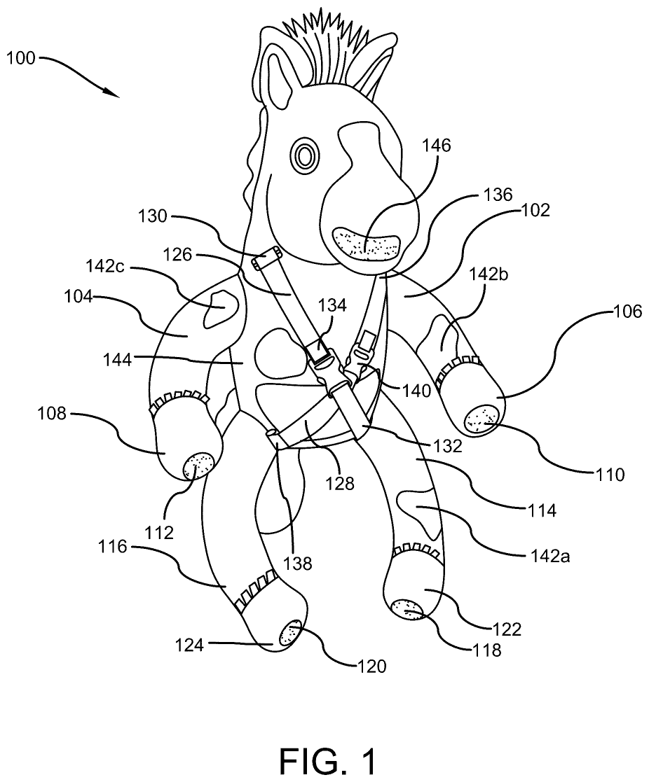

Referring initially to the drawings, FIG. 1 illustrates a front perspective view of the multifunctional sensory toy of the present invention in accordance with the disclosed structure. The multifunctional sensory toy 100 of the present invention is designed to aid individuals with sensory processing disorders (SPD) in achieving self-regulation through various sensory strategies. In the present embodiment, the multifunctional sensory device 100 is in the form of a plush animal and is designed as engaging and non-threatening for users. However, the device 100 may be in any form that resembles any animal, cartoon character, fictional character, human, etc.

The device 100 can come in different forms for older children and adults and may resemble a standard backpack, purse, bag, etc. for more discrete usage. More specifically, the multifunctional sensory toy 100 preferably includes a pair of arms 102,104 which are adapted to removably attach to each other at the distal ends 106,108 thereof. The distal ends 106,108 have hook and loop fasteners 110,112 disposed thereon for allowing a user to use the toy 100 as a backpack, wherein the arms 102,104 are secured around a user's torso/upper body. The fasteners 110,112 may be any other fastener type such as but not limited to snap buttons, buckle, magnets, etc.

The toy 100 includes a pair of legs 114,116 with each leg 114,116 includes a fastener 118,120 at the bottom end 122,124 thereof for allowing the legs to removably attach to each other. The fasteners 118,120 may be any fastener type such as but not limited to hook and loop, snap button, buckle, magnets, etc.

For allowing the toy 100 to be used as a backpack, the toy 100 includes at least one pair of belts or shoulder straps 126,128. More specifically, a first belt 126 extends from one shoulder 130 area of the device 100 to the diagonally opposite hip 132 area. The first belt 126 includes a first buckle 134 (or another fastener such as, but not limited to, snap button, hook and loop, etc.) for fastening the first belt 126. Similarly, a second belt 128 extends from the other shoulder 136 to the diagonally opposite hip 138. The second belt 128 also includes a corresponding buckle 140 (or another fastener such as but not limited to snap button, hook and loop, etc.) for fastening and adjusting the belt 128. The thick shoulder belt straps 126,128 may provide compression, which can be comforting and help with self-regulation for the SPD user.

The toy 100 may also include a plurality of removable weights 142a-c which can be removably added to the toy 100 to provide pressure, which is calming for individuals with SPD. The weights 142a-c can vary in size and weight and provide pressure on different body parts of a user carrying the toy 100 depending upon the position of the weights 142a-c within the toy 110.

The toy 100 preferably includes different textures such as but not limited to arms 102,104 that include rough texture, legs 114,116 that include soft texture, and a torso 144 that includes stringy texture (or any combination or variation thereof). Different textures provide various tactile sensory sensations for people with SPD and in one embodiment, the textures can be customized and interchangeable based on individual preference.

At least one scent emitter 146 is included in the toy 100 and is used to provide calming or alerting smells to the user. The smell dispensed by the emitter 146 is preferably natural and can be one or more from Lavender, Chamomile, Peppermint, Cinnamon, or any other calming scent, alerting scent, or neutral scent. The scent emitter 146 can be placed in a pouch or can be clipped on to the toy 100.

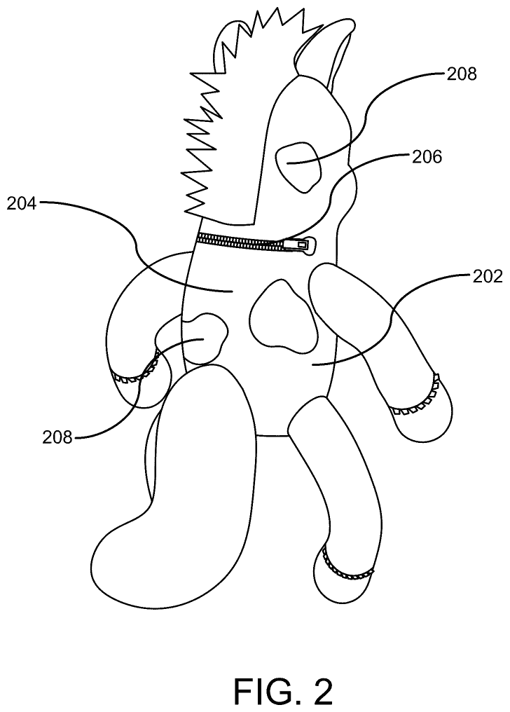

FIG. 2 illustrates a rear perspective view showing the back side of the multifunctional sensory toy of the present invention in accordance with the disclosed structure. As illustrated, the rear surface 202 of the toy 100 includes at least one zippered pocket 204 which is secured using a fastener 206 or another fastener such as, but not limited to, a zipper, hook and loop, snap button, etc. The zipper 206 provides secure storage of personal items and other sensory items. The rear surface 202 also may include at least one removable weighted pouches 208 such as sand-filled pouches, bead-filled pouches, weighted discs, and more to help individuals with sensory processing disorders feel more grounded and calmer when wearing the toy 100.

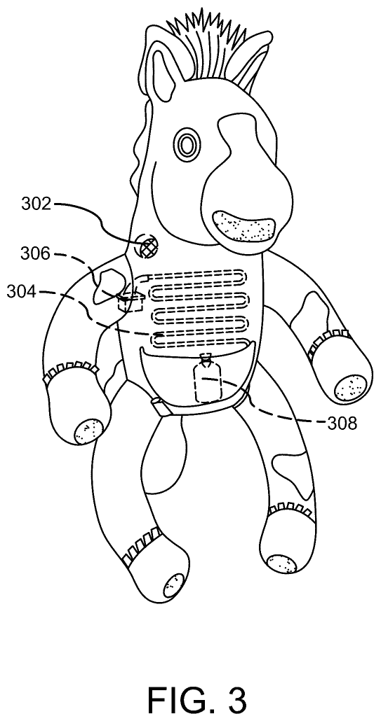

FIG. 3 illustrates another perspective view of the multifunctional sensory toy of the present invention showing internal components in accordance with the disclosed structure. As illustrated, the toy 100 includes a Bluetooth speaker 302 for producing calming sounds for the SPD individual wearing the toy 110. The speaker 302 can be wirelessly connected to a remote device such as a smartphone for receiving audio from the smartphone for playing. At least one vibration coil 304 is embedded inside the toy 100 for producing gentle vibrations for tactile stimulation while a user is wearing the toy 110. The intensity of vibrations can be adjusted using an electric switch 306 (and or any control of the like) as per the preferences of an individual. The electric switch 306 is adapted to provide electric power to both the Bluetooth speaker 302 and the vibration coil 304. Different calming items 308 such as a heat bag can be placed in different pockets 310 disposed in the toy 100.

The vibration coil 304 is adapted to measure the vitals of a user carrying the toy 100 on the back thereof and accordingly via at least one sensor 305, automatically adjusts the vibrations generated therefrom for calming the user. The toy 100 can send a notification to the connected remote smart device when the vitals of the user carrying the toy 100 go above a predetermined threshold. The vibration coil 304 is also adapted to provide heat to the user for providing a soothing effect.

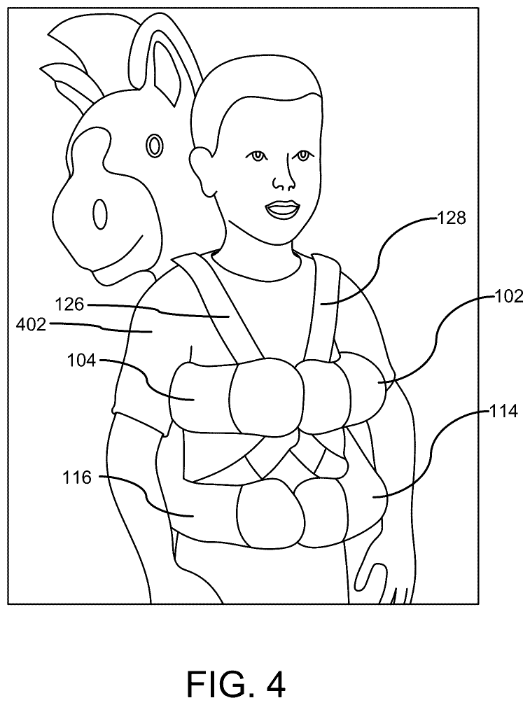

FIG. 4 illustrates a perspective view of a user carrying the multifunctional sensory SPD device of FIG. 1 as a backpack in accordance with the one embodiment of the present invention. The toy 100 can be carried as a backpack and is secured to the user 402 using the shoulder straps 126,128. Additional support is provided by attaching the arms 102,104 using the fasteners 110,112 and attaching the legs 114,116 using the fasteners 118,120. It will be apparent to a person skilled in the art that the multifunctional sensory SPD device 100 provides multiple sensory inputs, such as weighted compression combined with vibration or soothing sounds where the sensory inputs adapt based on user interaction or feedback. The device 100 is foldable for easy storage and transport and the device is easy to clean and maintain, with removable and washable covers or inserts.

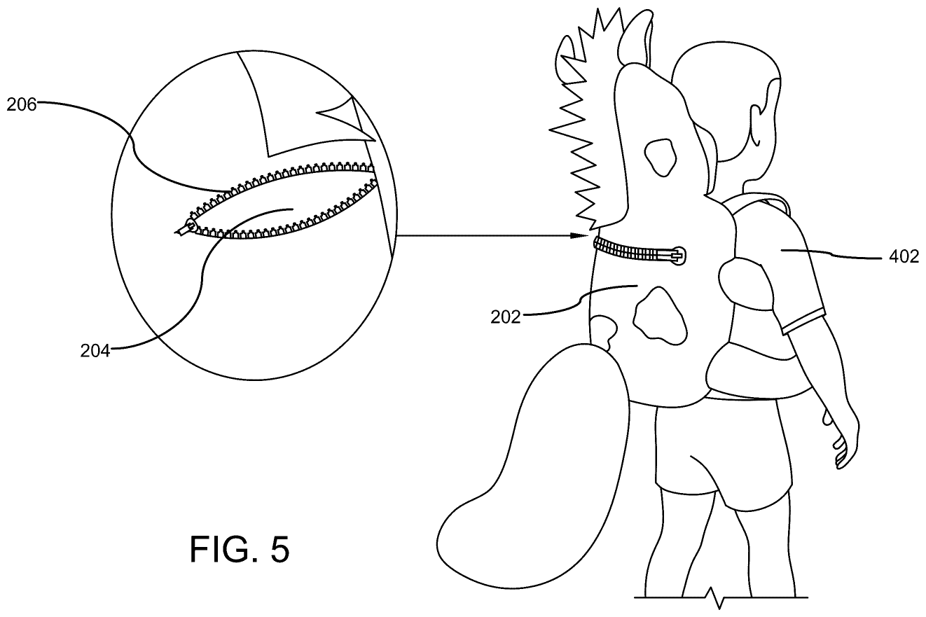

FIG. 5 illustrates a rear perspective view showing the user carrying the sensory toy as a backpack in accordance with the disclosed structure. When the toy 100 is carried as a backpack, the toy 100 remains secured to the back 502 of the user 402. The zippered pocket 204 can be used for storing personal items, electronic equipment, medicines, and more. In the preferred embodiment, the toy 100 is modular and different components such as legs, arms, and other internal components can be detached and replaced. The toy 100 can come in different sizes, designs, colors, and shapes that create a range of characters or animals that are visually appealing and can be marketed as a collectible series.

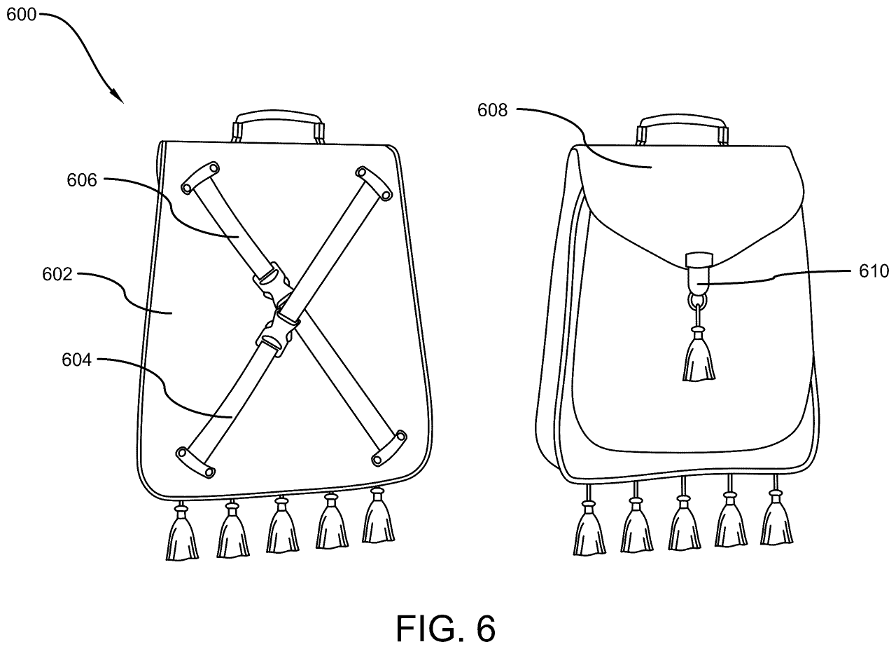

FIG. 6 illustrates a perspective view of another embodiment of the multifunctional sensory device of the present invention in accordance with the disclosed structure. The sensory device 600 is designed for adults and is preferably in the form of a backpack with the rear surface 602 having a pair of shoulder straps 604,606 for carrying the device 600. The device 600 includes all the features of the device 100 as described above and can be customized to meet the requirements of adult users. The device 600 includes interchangeable weights, textures, scent emitters, and a speaker. The device 600 incorporates Bluetooth connectivity to monitor the user's sensory responses and provide feedback to caregivers or therapists. The device 100 can include a cover 608 for securing the items stored therein and may include a buckle 610 for secure storage.

The sensory devices of different embodiments of the present invention combine sensory inputs including but not limited to tactile, olfactory, auditory, and proprioceptive that help users regulate their sensory processing. The device helps to improve focus, reduce anxiety, and better overall functioning.

The coil 304 and speaker may be powered by at least one battery 310. The battery 310 may be disposable or rechargeable.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “multifunctional sensory SPD device”, “multifunctional sensory toy”, “multifunctional sensory device”, “toy”, and “device” are interchangeable and refer to the customizable sensory device for managing sensory processing disorders 100,600 of the present invention.

Notwithstanding the forgoing, the customizable sensory device for managing sensory processing disorders 100,600 of the present invention can be of any suitable size and configuration as is known in the art without affecting the overall concept of the invention, provided that it accomplishes the above-stated objectives. One of ordinary skill in the art will appreciate that the customizable sensory device for managing sensory processing disorders 100,600 as shown in the FIGS. are for illustrative purposes only, and that many other sizes and shapes of the customizable sensory device for managing sensory processing disorders 100,600 are well within the scope of the present disclosure. Although the dimensions of the customizable sensory device for managing sensory processing disorders 100,600 are important design parameters for user convenience, the customizable sensory device for managing sensory processing disorders 100,600 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A multifunctional sensory SPD device comprising:
a body configured to resemble an animal, wherein the body includes a pair of arms and a pair of legs;
a first fastener pair positioned on a first distal end of the pair of arms;
a second fastener pair position on a second distal end of the pair of legs, wherein the pair of legs attached to the pair of arms via the first fastener pair and the second fastener pair;
a pair of shoulder straps extending from the body, wherein the shoulder straps are adjustable and comprised of a fastening mechanism;
a removable weight disposed within the body configured to apply pressure to a user wearing the multifunctional sensory SPD device; and
a scent emitter integrated into the body that dispenses a scent.

2. The multifunctional sensory SPD device of claim 1, wherein the first fastener pair and the second fastener pair are comprised of a hook fastener and a loop fastener.

3. The multifunctional sensory SPD device of claim 1, wherein the first fastener pair and the second fastener pair are comprised of a snap button fastener or a buckle.

4. The multifunctional sensory SPD device of claim 1, wherein the body is in the form of a plush animal.

5. The multifunctional sensory SPD device of claim 1, wherein the scent is comprised of a lavender, a chamomile, a peppermint, or a cinnamon.

6. The multifunctional sensory SPD device of claim 1, wherein the pair of arms are comprised of a first texture and the pair of legs are comprised of a second texture.

7. A multifunctional sensory SPD device comprising:
a body comprised of a front surface and a rear surface;
a pocket positioned along the rear surface;
a removable weighted pouch integrated within the rear surface;
a Bluetooth speaker;
a vibration coil within the body that generates a vibration for a tactile stimulation; and
a control mechanism for adjusting an intensity of the vibration.

8. The multifunctional sensory SPD device of claim 7, wherein the vibration coil produces heat.

9. The multifunctional sensory SPD device of claim 7, wherein the pocket is comprised of a fastener.

10. The multifunctional sensory SPD device of claim 7, wherein the vibration coil is positioned within a torso of the body.

11. A multifunctional sensory SPD device comprising:
a body comprised of an arm and a leg, wherein the arm attaches to the leg;
an adjustable belt system;
a detachable weight disposed in a pocket;
an embedded scent emitter that dispenses a scent;
a Bluetooth speaker and a vibration coil that provides an auditory and a tactile stimulation; and
a sensor for monitoring a user's vitals and adjusting the tactile stimulation based on the user's vitals.

12. The multifunctional sensory SPD device of claim 11 further comprised of a heating bag.

13. The multifunctional sensory SPD device of claim 11, wherein the body resembles a backpack.

14. The multifunctional sensory SPD device of claim 11, wherein the body is washable.

15. The multifunctional sensory SPD device of claim 11 further comprised of a battery.

16. The multifunctional sensory SPD device of claim 11, wherein the body resembles an animal, a cartoon character, a fictional character, or a human.

17. The multifunctional sensory SPD device of claim 11 further comprised of a cover.

18. The multifunctional sensory SPD device of claim 11, wherein the arm is comprised of a rough texture.

19. The multifunctional sensory SPD device of claim 11, wherein the leg is comprised of a smooth texture.

20. The multifunctional sensory SPD device of claim 11, wherein the detachable weight is comprised of a weighted pouch.
